(19) United States
(12) Patent Application Publication
(10) Pub. No.: US 2018/0036217 A1
(43) Pub. Date: Feb. 8, 2018

(54) MOUSSE-TYPE COSMETIC COMPOSITION CONTAINING SCORIA

(71) Applicant: AMOREPACIFIC CORPORATION, Seoul (KR)

(72) Inventors: Hye Yeong JEON, Yongin-si (KR); Hae Jin JUNG, Yongin-si (KR); Jin Seob SHIN, Yongin-si (KR); Soon Ae AN, Yongin-si (KR); Byung Young KANG, Yongin-si (KR)

(73) Assignee: AMOREPACIFIC CORPORATION, Seoul (KR)

(21) Appl. No.: 15/555,840

(22) PCT Filed: Feb. 25, 2016
(86) PCT No.: PCT/KR2016/001876

§ 371 Date: Sep. 5, 2017

(30) Foreign Application Priority Data
Mar. 5, 2015 (KR) 10-2015-0030819

(51) Int. Cl. A61K 8/44; A61K 8/04; A61Q 19/00; A61K 8/96

(52) U.S. Cl. CPC A61K 8/44; A61K 8/965; A61K 8/046; A61Q 19/007; A61K 2800/52

(57) ABSTRACT

The present invention relates to a mousse-type cosmetic composition containing scoria and a neutralizing agent, having excellent formability since the composition swells and does not easily drip when discharged, and has low skin irritability.

### TECHNICAL FIELD

This application claims priority to and the benefit of Korean Patent Application No. 10-2015-0030819, filed on Mar. 5, 2015 in the Korean Intellectual Property Office, the disclosure of which is incorporated herein by reference in their entirety.

The present invention relates to a mousse-type cosmetic composition containing scoria, and more particularly, to a cosmetic composition capable of being used in the field of cosmetics which are applied to the human skin in order to contribute to beauty.

### BACKGROUND ART

The scientific name “scoria” is a volcanic stone made as clay burns due to high heat caused by volcanic activity, and thus is also known as a kind of natural ceramics. Scoria is found at Jeju-do, Korea, and is referred to as “Song-i” as the Jeju Island dialect. Scoria has physical properties: it has a slightly alkaline pH of approximately 7.2 to 7.8, and has various colors such as reddish brown, yellowish brown, black, and gray colors, depending on the mineral component generally contained as a vesicular texture.

It has been known that scoria contains a large amount of natural minerals, and thus has its own excellent physical properties such as a high far-infrared radiation rate, superior antibacterial activity, and excellent deodorization performance. In addition, Registered Korean Patent No. 1415996 filed by the applicant of the present invention discloses that scoria effectively inhibits the activity of 5α-reductase, which hypersecrets sebum in the sebaceous gland, by converting testosterone into dihydrotestosterone, which then binds to a receptor protein present in the cytoplasm to enter the nuclei, thereby activating sebaceous gland cells and promoting differentiation of the sebaceous gland cells.

Further, because scoria contains a large amount of natural minerals, the scoria tends to be highly usable for various beauty materials such as skin moisturizing, pore tightening, cleansing, antibacterial materials. Typically, scoria is ground into small particles and added for use as a material for cosmetics.

Among formulations made of a cosmetic composition, mousse formulations which may be sprayed in the form of foam and spread on the skin and have a light sense of feeling in use have come into the spotlight.

Accordingly, a low-viscosity liquid formulation including a propellant was prepared as a cosmetic composition in order to obtain a mousse formulation containing scoria, but it has a problem in that scoria may precipitate due to low stability of the cosmetic composition itself. Thus, to develop a mousse formulation in which a cosmetic composition containing scoria may be uniformly sprayed and discharged, stabilization of the cosmetic composition should be preferentially required.

Meanwhile, neutralizing agents for neutralization have been used in the cosmetic composition for mousse formulations. Among these, an amine-based neutralizing agent has been most widely used. However, the amine-based neutralizing agent causes skin irritation-related problems, and also serves to prevent the cosmetic composition from being formulated into a mousse phase, that is, hinder formability of a mousse formulation.

Therefore, a neutralizing agent which has no skin irritability and does not interfere with the formability of the mousse formulation should be preferentially developed to prepare a mousse formulation containing scoria.

### PRIOR-ART DOCUMENT

Cosmetic composition containing scoria (Korean Unexamined Patent Publication No. 10-2013-0001441)

### DISCLOSURE

### Technical Problem

Therefore, it is an object of the present invention to provide a mousse-type cosmetic composition containing scoria, which is able to solve a problem of not uniformly dispersing scoria in consideration of an innate phase and physical properties of scoria and has low skin irritability and an improved formability of a mousse formulation.

### Technical Solution

Therefore, the present invention is designed to solve the problems of the prior art, and it is an object of the present invention to provide a mousse-type cosmetic composition including scoria and a compound represented by the following Formula 1 as a neutralizing agent:

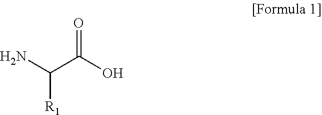

wherein R₁ is as described in this specification.

The neutralizing agent includes arginine, glycine, alanine, valine, leucine, isoleucine, serine, threonine, aspartic acid, glutamic acid, lysine, ornithine, cysteine, methionine, histidine, phenylalanine, tyrosine, and tryptophane.

Preferably, the neutralizing agent is L-arginine, and may be included at 0.4 to 3% by weight, based on the total weight of the cosmetic composition.

Also, the mousse-type cosmetic composition of the present invention may further include water, a polyol, a thickening agent, and a fatty acid. Preferably, in the mousse-type cosmetic composition, the scoria, the neutralizing agent, the water, the polyol, the thickening agent, and the fatty acid may be included at 0.5 to 10% by weight, 0.4 to 1% by weight, 70 to 90% by weight, 5 to 10% by weight, 0.5 to 4% by weight, and 3 to 10% by weight, respectively.

### Advantageous Effects

The mousse-type cosmetic composition containing scoria according to the present invention has excellent formability because the cosmetic composition easily swells and does not easily drip when a mousse formulation is discharged, and also has a property of uniformly dispersing scoria.

Also, the cosmetic composition may satisfy the requirements such as low irritability, which is one of essential characteristics in the field of cosmetics, because the cosmetic composition has excellent safety to irritation on the skin.

### DESCRIPTION OF DRAWINGS

FIG. 1 is an image of mousse-type cosmetic compositions prepared in Example 1 and Comparative Examples 1 and 2 being applied onto the skin.

FIG. 2A is an image of carbon black being applied onto a surface of the skin, and FIG. 2B is an image of the mousse-type cosmetic composition of Example 1 after the cosmetic composition is applied onto the skin and the skin is washed with water.

### BEST MODE

Hereinafter, the detailed description of the present invention will be provided with reference to certain embodiments, the present invention of which can be put into practice. In this case, the present invention should be described in such sufficient detail that a person having ordinary skill in the art can put the present invention put into practice. It should be understood that various embodiments of the present invention may be different from each other, but may not be mutually exclusive to each other. Therefore, it should be appreciated that the detailed description provided below is not interpreted as having a limited meaning, but the scope of the present invention should be recognized to encompass all the contents disclosed in the claims, and equivalents thereof.

A mousse-type cosmetic composition according to one exemplary embodiment of the present invention may include scoria, a polyol, a thickening agent, a fatty acid, and a neutralizing agent. Hereinafter, each of the components will be described in detail.

<Scoria>

The scientific name “scoria” of the present invention refers to a porous basaltic rock in which a volume of pores is similar to that of solids. Scoria includes volcanic ashes, volcanic gravel, volcanic rock, and volcanic bombs. In Korean, scoria is produced at Jeju-do, and is known as a natural material having a far-infrared radiation rate of 92 to 93%, and a deodorization rate of 97% in a natural state.

The scoria produced at Jeju-do has physical properties: it has a melting point of 1,120 to 1,210° C. and an absorption rate of 17.7 to 32.5% with respect to its mass. Also, scoria has various colors depending on the contents of materials contained in the scoria. In this case, scoria having a high content of silicon oxide has a dark gray color, scoria having a high content of aluminum oxide has a yellowish brown or black color, and scoria having a high content of titanium oxide and iron oxide has a reddish brown color. In addition, scoria may contain various minerals such as calcium oxide, magnesium oxide, potassium oxide, sodium oxide, etc.

The scoria of the present invention may be obtained using methods generally known in the related art, and a method of preparing scoria is not particularly limited. Specifically, the scoria of the present invention may be prepared by collecting crude scoria, cleansing the crude scoria to remove impurities, grinding the crude scoria into powder using a grinding machine, heating the powder to a temperature of 100° C. or higher, and applying a pressure to the powder.

Such scoria is used in a powdery phase. In this case, scoria having an average particle diameter used in the field of cosmetics is used herein. Typically, because a specific surface area of scoria increases with a decreasing particle diameter, the scoria has an enhanced moisturizing effect, etc. On the other hand, because a distance between particles of scoria becomes closer with an increasing specific surface area, the scoria has an improved an antibacterial rate, etc. However, the particle diameter of scoria needs to be limited so that the scoria is uniformly dispersed in a foamed mousse composition when prepared into a mousse formulation. In this case, when the particle diameter is very small, aggregation between powders may occur. On the other hand, when the particle diameter is very high, precipitation may occur. Preferably, scoria having a particle diameter of approximately 0.5 to 100 μm, preferably approximately 1 to 50 μm is used in the present invention. In this case, the scoria powder may be obtained using any conventional methods known in the related art, such as a dry grinding method, a wet grinding method, etc.

The scoria according to the present invention is used at a content of 0.5 to 10% by weight, preferably 0.5 to 7% by weight, based on the total weight of the mousse-type cosmetic composition. When the content is less than this content range, effects expected from the use of scoria, such as skin moisturizing, pore tightening, cleansing, and antibacterial efficiencies and effects, may be degraded. On the other hand, when the content is greater than this content range, an excessive amount of scoria may be problematic due to an increased characteristic of adsorbing oils.

<Polyol>

The polyol of the present invention refers to a polyhydric alcohol, and may, for example, selected from glycerin, 1,3-butylene glycol, propylene glycol, polyethylene glycol, sorbitol, and a mixture thereof. Preferably, a mixture of 1,3-butylene glycol and glycerin may be used as the polyol, but the present invention is not limited thereto. In this case, all the polyol components for cosmetics may be applied to the present invention.

The polyol according to the present invention is used to realize a moisturizing effect. In this case, the polyol is used at a content of 5 to 10% by weight, preferably 6 to 9% by weight, based on the total weight of the mousse-type cosmetic composition, in order to aid in dissolving the other components which are insoluble in water. When the content is less than this content range, moisturizing feeling, a moisturizing effect, and stability expected from the use of polyol may not sufficiently secured. On the other hand, when the content is greater than this content range, foam may not be formed when the composition is discharged from a product-manufacturing device. Therefore, the polyol may be properly used in this content range.

<Thickening Agent>

The thickening agent of the present invention refers to a component that has a characteristic of increasing fluidity or promoting rearrangement due to the polarity of water when dispersed in water. For example, the thickening agent may include one selected from bentonite, sodium magnesium silicate, magnesium silicate, magnesium aluminum silicate, xanthan gum, Carbomer, Sepigel, and hydroxyethyl cellulose, but the present invention is not limited thereto.

Preferably, as a natural inorganic thickening agent, bentonite may be used. Bentonite serves as a viscosity adjuster to increase the viscosity of an emulsion. Therefore, the bentonite is used to enhance the viscosity of an emulsion and improve the stability of the emulsion when prepared into cosmetics such as a cream, a lotion, a makeup composition, etc. Also, the bentonite serves as a dispersion stabilizing agent by forming and stabilizing a lattice structure at a phase interface between an oily phase and an aqueous phase which are not miscible with each other, aids in reducing an amount of a surfactant, and serves to reduce stickiness of a polymeric material.

Bentonite has a property of absorbing an extra amount of sebum because it is porous. Therefore, when the bentonite is applied to an acne-prone skin or an oily skin, the bentonite may serve to absorb oils to prevent greasiness in the skin. Also, because the bentonite functions o adsorb oils, the bentonite is used in products in which a large amount of oil needs to be inevitably used, thereby slightly reducing a sense of feeling oily and giving a refreshing sense of feeling in use.

The thickening agent according to the present invention is used at a content of 0.5 to 4% by weight, preferably 1 to 3% by weight, based on the total weight of the mousse-type cosmetic composition. When the content is less than this content range, foam may not be formed due to insufficient viscosity when the composition is discharged from a product-manufacturing device. On the other hand, when the content is greater than this content range, the size of foam may be rather reduced due to an excessive increase in viscosity. Therefore, the thickening agent may be properly used in this content range.

<Fatty Acid>

The fatty acid of the present invention includes an unsaturated fatty acid and a saturated fatty acid, both of which are used in the cosmetics. For example, the fatty acid of the present invention may include one selected from the group consisting of palmitic acid, stearic acid, lauric acid, caproic acid, heptanoic acid, caprylic acid, capric acid, undecanoic acid, tridecanoic acid, pentadecanoic acid, arachidic acid, behenic acid, myristic acid, isostearic acid, oleic acid, linoleic acid, linolenic acid, and a combination thereof, but the present invention is not limited thereto.

The fatty acid according to the present invention is used to realize stability of the cosmetic composition. In this case, the fatty acid is used at a content of 3 to 10% by weight, preferably 3 to 9% by weight, based on the total weigh of the mousse-type cosmetic composition. When the content is less than this content range, the phenomena such as precipitation, and the like may occur due to degraded stability of the cosmetic composition. On the other hand, when the content is greater than this content range, there is no significant advantage in terms of effects, and it is uneconomical. Therefore, the fatty acid may be properly used in this content range.

<Neutralizing Agent>

In particular, the neutralizing agent of the present invention serves to neutralize the fatty acid with an amino group, and also to reduce skin irritability and enhance formability of a mousse formulation. The term “formability” of the mousse formulation disclosed in this specification refers to a situation in which a cosmetic composition does not flow downward, and swells upward from a contact surface of the skin while maintaining a constant viscosity when the cosmetic composition is discharged out of a container.

Preferably, the neutralizing agent is a compound represented by the following Formula 1:

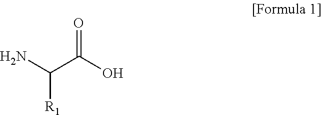

wherein R₁ includes one selected from the group consisting of H, —CH₃, —CH(CH₃)₂, —CH₂CH(CH₃)₂, —CH(CH₃)CH₂CH₃, —CH₂OH, —CH(OH)CH₃, —CH₂COOH, —CH₂CHCOOH, —(CH₂)₄NH₂, —(CH₂)₃NH₂,

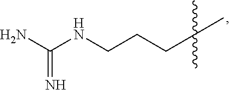 [SMILES: *CCCNC(=N)N]

—CH₂SH, —(CH₂)₂SCH₃,

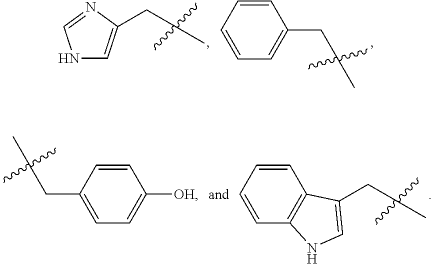

The neutralizing agent contains two carbon atoms and has an amino group (—NH₂) and R₁ substituted therein. The neutralizing agent included in the present invention has low skin irritability, compared to primary and tertiary amines (for example, tromethamine and triethylamine) or secondary amines as generally used in the art, and is suitable for mousse formulations.

Hereinafter, examples of R₁ to be substituted in the following Formula 1 are listed, and the compounds that may be included in the neutralizing agent will be described in detail, as listed in Table 1.

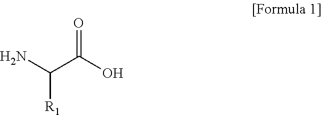

TABLE 1

| Neutralizing agent | Formula | R₁ |
|---|---|---|
| L-Arginine | 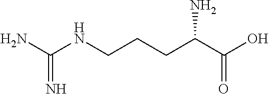 [SMILES: N=C(N)NCCC[C@H](N)C(=O)O] | 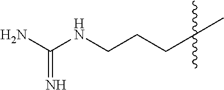 [SMILES: *CCCNC(=N)N] |
| Glycine | 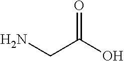 | —H |
| Alanine | 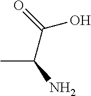 | —CH₃ |
| Valine | 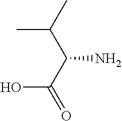 | —CH(CH₃)₂ |
| Leucine | 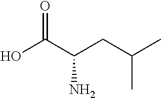 | —CH₂CH(CH₃)₂ |
| Isoleucine | 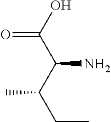 | —CH(CH₃)CH₂CH₃ |
| Serine | 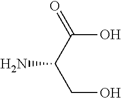 | —CH₂OH |
| Threonine | 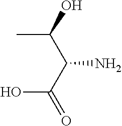 | —CH(OH)CH₃ |
| Aspartic acid | 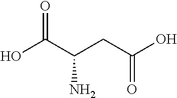 | —CH₂COOH |
| Glutamic acid | 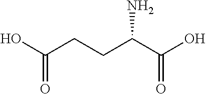 | —(CH₂)₂COOH |
| Lysine | 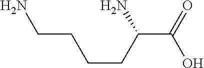 | —(CH₂)₄NH₂ |
| Ornithine | 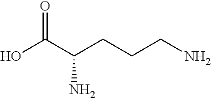 | —(CH₂)₃NH₂ |
| Cysteine | 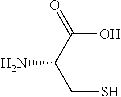 | —CH₂SH |
| Methionine | 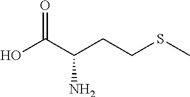 | —(CH₂)₂SCH₃ |
| Histidine | 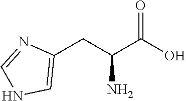 [SMILES: N[C@@H](Cc1c[nH]cn1)C(=O)O] | 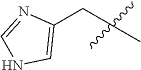 [SMILES: *Cc1c[nH]cn1] |
| Phenylalanine | 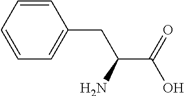 [SMILES: N[C@@H](Cc1ccccc1)C(=O)O] | 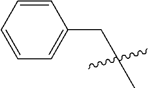 [SMILES: *Cc1ccccc1] |
| Tyrosine | 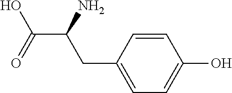 [SMILES: N[C@@H](Cc1ccc(O)cc1)C(=O)O] | 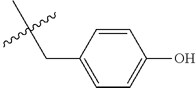 [SMILES: *Cc1ccc(O)cc1] |
| Tryptophane | 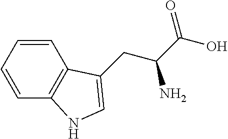 [SMILES: N[C@@H](Cc1c[nH]c2ccccc12)C(=O)O] | 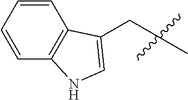 [SMILES: *Cc1c[nH]c2ccccc12] |

As listed in Table 1, the neutralizing agent represented by Formula 1 serves to stabilize scoria and neutralize a composition in which water, a polyol, a thickening agent, and a fatty acid are mixed, and simultaneously to secure formability of these mousse formulations. Also, the neutralizing agent of the present invention has low skin irritability, compared to the primary, secondary and tertiary amines often used as the neutralizing agent in the cosmetic composition.

The content of such a neutralizing agent of Formula 1 may vary depending on the content of the fatty acid. For example, the neutralizing agent is used at a content of 0.4 to 3% by weight, preferably 0.4 to 1% by weight, based on the total weight of the cosmetic composition. When the content is less than this content range, the formability of mousse formulations may be highly degraded. On the other hand, when the content is greater than this content range, foam may not be properly formed. Therefore, the neutralizing agent may be properly used in this content range.

In particular, L-arginine is preferably used as the neutralizing agent according to the present invention. L-arginine is an amino acid existing in all organisms, and thus has no skin irritability, promotes production of epithelial cells, is a precursor for forming L-proline as a factor important for biosynthesis of collagen, and is effective in healing skin wounds.

The mousse-type cosmetic composition according to the present invention may be prepared into products used for various purposes such as skin moisturizing, pore tightening, cleansing, and antibacterial purposes, etc. In this case, the mousse-type cosmetic composition may further include known components in addition to the aforementioned components.

For example, the mousse-type cosmetic composition may further include, as the components to be added, an inorganic solvent, a dissolving agent, a concentrating agent, a gelling agent, a softening agent, an antioxidant, a suspending agent, a stabilizing agent, a foaming agent, a flavoring agent, a surfactant, water, an ionic or non-ionic emulsifying agent, a filler, a chelating agent, a preservative, a vitamin, blocking agent, a moisturizing agent, an essential oil, a dye, a pigment, a hydrophilic or lyphophilic activating agent, a lipid vesicle, or adjuvants generally used in the cosmetics.

### [Mode for Invention]

Hereinafter, the mousse-type cosmetic composition of the present invention will be described in detail with reference to examples of the present invention. However, it should be understood that these examples disclosed herein are not intended to limit the scope of the present invention.

### EXAMPLE AND COMPARATIVE EXAMPLES

### Preparation of Mousse-Type Cosmetic Compositions

The types and contents (% by weight) of water, a polyol, a thickening agent, scoria, and a fatty acid other than a neutralizing agent were set as listed in the following Table 2, and the neutralizing agent was added at different contents to prepare mousse-type cosmetic compositions.

TABLE 2

| | | | | | (Converted |
|---|---|---|---|---|---|
| | | | | | to % by |
| | | Comparative | Comparative | | weight for |
| Compositions (parts by weight) | | Example 1 | Example 2 | Example 1 | Example 1) |
| Water | Purified water | 100 | 100 | 100 | 81.4 |
| Polyol | Glycerin and | 10.00 | 10.00 | 10.00 | 8.14 |
| | butylene glycol | | | | |
| | mixed at ratio of | | | | |
| | 3:7 | | | | |
| Thickening | Bentonite | 2.00 | 2.00 | 2.00 | 1.6 |
| agent | | | | | |
| Scoria | Ground scoria | 3.00 | 3.00 | 3.00 | 2.44 |
| Fatty acid | Stearic acid and | 7.00 | 7.00 | 7.00 | 5.7 |
| | palmitic acid mixed | | | | |
| | at ratio of 6:4 | | | | |
| Neutralizing | Tromethamine | 0.65 | 0 | 0 | — |
| agent | Triethylamine | 0 | 0.81 | 0 | — |
| | L-arginine | 0 | 0 | 0.80 | — |

A method of preparing each of the mousse-type cosmetic compositions of Example and Comparative Examples, which included the components as listed in Table 2, was performed, as follows:

(1) Polyol-containing purified water and a fatty acid-containing oil phase portion were mixed at the contents as listed in Table 2, and then heated to a temperature of 65° C. to 85° C.

(2) The resulting mixture was stirred at 7,500 rpm by a mixer to be emulsified.

(3) A thickening agent and scoria were added, and then dispersed while stirring at 5,000 rpm using the mixer.

(4) A rotation speed of the mixer was reduced to 3,000 rpm, and a neutralizing agent was slowly added to neutralize the mixture. Thereafter, the temperature was reduced to 30° C. to prepare the cosmetic compositions of Example and Comparative Examples.

The neutralizing agent used in Comparative Example 1 was triethylamine that was a primary amine having a polyhydric alcohol group, and the neutralizing agent used in Comparative Example 2 was triethylamine that was a tertiary amine. In Example 1, L-arginine was used as the neutralizing agent according to one exemplary embodiment of the present invention. In this case, the neutralizing agent was slowly added until all the cosmetic compositions of Example and Comparative Examples finally prepared in step (4) had the same neutralization rate.

### Experimental Example 1

### Measurement of Formability of Mousse Formulation

To determine whether the cosmetic compositions prepared in Example 1 and Comparative Examples 1 and 2 had the formability of mousse formulations, each of the cosmetic compositions was discharged onto a surface of a human skin, and the formability and flowability were measured.

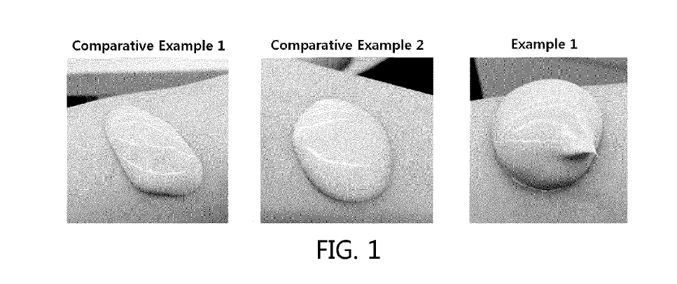

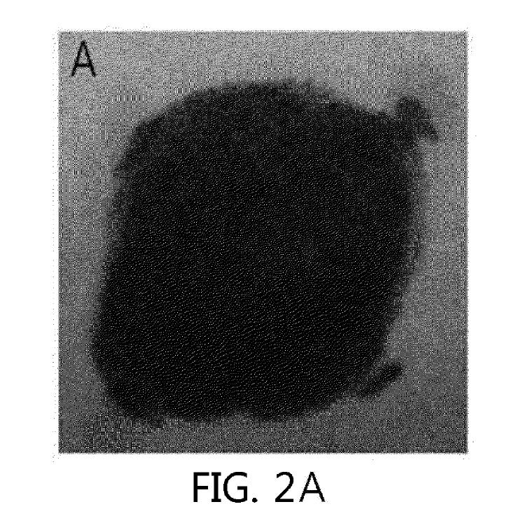

FIG. 1 is an image of mousse-type cosmetic compositions prepared in Example 1 and Comparative Examples 1 and 2 being applied onto the skin.

Referring to FIG. 1, it can be seen that the cosmetic composition of Comparative Example 1 in which triethylamine was used as the neutralizing agent had almost no formability in which a mousse formulation did not swell when discharged, and easily flowed down after the cosmetic composition was applied onto the skin.

Also, it can be seen that the cosmetic composition of Comparative Example 2 in which triethylamine was used as the neutralizing agent had slightly improved formability in which a mousse formulation swelled when discharged, compared to the cosmetic composition of Comparative Example 1, but flowed down after the cosmetic composition was applied onto the skin, the property of which was not greatly different from that of Comparative Example 1.

It can be seen that the cosmetic composition of Example 1 of the present invention, in which L-arginine was used as the neutralizing agent, had good formability in which a mousse formulation swelled when discharged, and did not flow down on a surface of the skin after the cosmetic composition was applied onto the skin. Therefore, it can be seen that the cosmetic composition played a role as the neutralizing agent, and also had an effect of improving the formability of the mousse formulation when the neutralizing agent of the present invention was added and used.

### Experimental Example 2

### Measurement of Skin Irritability

To check degrees of skin irritability of the mousse-type cosmetic compositions prepared in Example 1 and Comparative Examples 1 and 2, an experiment was performed, as follows.

For a panel of 10 cosmetics experts, the cosmetic compositions of Example 1 and Comparative Example 1 were uniformly spread on skin surfaces of the cosmetics experts, and the number of the cosmetics experts who felt irritable was then checked. The results are listed in the following Table 3.

TABLE 3

| | Comparative | Comparative | |
|---|---|---|---|
| Items | Example 1 | Example 2 | Example 1 |
| Number of cosmetics | 3 | 6 | None |
| experts who feel | | | |
| irritable | | | |

As disclosed in Experimental Example 1, it can be seen that the cosmetic composition of Comparative Example 2 had improved formability, compared to the cosmetic composition of Comparative Example 1. However, from the results of Experimental Example 2, it can be seen that that both of the cosmetic compositions had strong skin irritability.

On the other hand, the cosmetic composition of Example 1 of the present invention had improved formability of mousse formulations, and there was no panel of cosmetics experts who felt irritable on the skin.

### Experimental Example 3

### Measurement of Contaminant Removal Efficiency

To check an ability of the mousse-type cosmetic composition of Example 1 of the present invention to remove fine dust, an experiment was performed, as follows.

This experiment was carried out by applying carbon black, which had a particle diameter similar to fine dust giving direct irritation to the skin but was harmless to the skin, onto the skin instead of the fine dust. Specifically, carbon black was applied onto the skin, and the mousse-type cosmetics of Example 1 were again applied onto the skin, and kept for 10 minutes. Thereafter, the skin was washed with running water, and a degree of removal of fine dust was then determined.

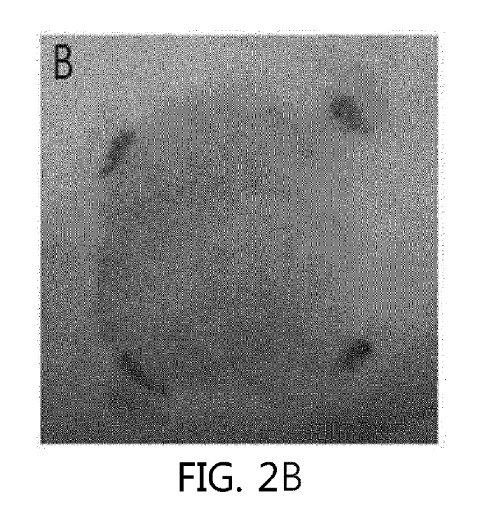

FIG. 2A is an image of carbon black being applied onto a surface of the skin, and FIG. 2B is an image of the mousse-type cosmetic composition of Example 1 after the cosmetic composition is applied onto the skin and the skin is washed with water. Referring to FIG. 2A, it can be seen that the fine dust was uniformly dispersed on a surface of the skin. Referring to FIG. 2B, it can be seen that most of the carbon black was removed.

From these results, it can be seen that the scoria-containing mousse-type cosmetic composition according to the present invention had an excellent ability to remove fine dust accumulated on the skin surface as one of skin contaminants because foam was compactly maintained after the cosmetic composition was discharged.

1. A mousse-type cosmetic composition comprising:
scoria and
a compound represented by the following Formula 1 as a neutralizing agent: 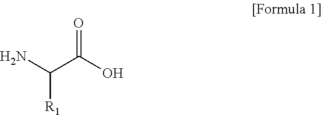
wherein R₁ comprises one selected from the group consisting of H, —CH₃, —CH(CH₃)₂, —CH₂CH(CH₃)₂, —CH(CH₃)CH₂CH₃, —CH₂OH, —CH(OH)CH₃, —CH₂COOH, —CH₂CHCOOH, —(CH₂)₄NH₂, —(CH₂)₃NH₂, 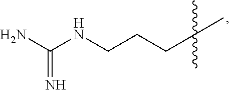 [SMILES: *CCCNC(=N)N]
—CH₂SH, —(CH₂)₂SCH₃,

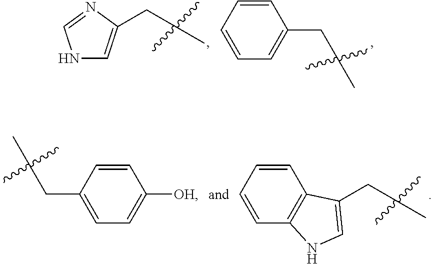

2. The mousse-type cosmetic composition of claim 1, wherein the neutralizing agent comprises one selected from the group consisting of arginine, glycine, alanine, valine, leucine, isoleucine, serine, threonine, aspartic acid, glutamic acid, lysine, ornithine, cysteine, methionine, histidine, phenylalanine, tyrosine, tryptophane, and a combination thereof.

3. The mousse-type cosmetic composition of claim 1, wherein the neutralizing agent is L-arginine, and included at 0.4 to 3% by weight, based on the total weight of the cosmetic composition.

4. The mousse-type cosmetic composition of claim 1, wherein the scoria is included at 0.5 to 10% by weight, based on the total weight of the cosmetic composition.

5. The mousse-type cosmetic composition of claim 1, wherein the mousse-type cosmetic composition further comprises water, a polyol, a thickening agent, and a fatty acid.

6. The mousse-type cosmetic composition of claim 5, wherein the cosmetic composition comprises the scoria at 0.5 to 10% by weight, the neutralizing agent at 0.4 to 1% by weight, the water at 70 to 90% by weight, the polyol at 5 to 10% by weight, the thickening agent at 0.5 to 4% by weight, and the fatty acid at 3 to 10% by weight, based on the total weight of the cosmetic composition.

7. The mousse-type cosmetic composition of claim 5, wherein the polyol comprises one selected from the group consisting of glycerin, 1,3-butylene glycol, propylene glycol, polyethylene glycol, sorbitol, and a combination thereof.

8. The mousse-type cosmetic composition of claim 5, wherein the thickening agent comprises one selected from the group consisting of bentonite, sodium magnesium silicate, magnesium silicate, magnesium aluminum silicate, xanthan gum, Carbomer, Sepigel, hydroxyethyl cellulose, and a combination thereof.

9. The mousse-type cosmetic composition of claim 5, wherein the fatty acid comprises one selected from the group consisting of palmitic acid, stearic acid, lauric acid, caproic acid, heptanoic acid, caprylic acid, capric acid, undecanoic acid, tridecanoic acid, pentadecanoic acid, arachidic acid, behenic acid, myristic acid, isostearic acid, oleic acid, linoleic acid, linolenic acid, and a combination thereof.
